(19) United States
(12) Patent Application Publication
(10) Pub. No.: US 2014/0276591 A1
(43) Pub. Date: Sep. 18, 2014

(54) PERCUTANEOUS INTERVERTEBRAL ANNULAR REGENERATION

(71) Applicant: MI4SPINE, LLC, Bloomfield Village, MI (US)

(72) Inventors: Miguelangelo J. Perez-Cruet, Bloomfield, MI (US); John R. Pepper, Cheshire, CT (US)

(73) Assignee: MI4SPINE, LLC, Bloomfield Village, MI (US)

(21) Appl. No.: 14/211,802

(22) Filed: Mar. 14, 2014

Related U.S. Application Data
(60) Provisional application No. 61/783,935, filed on Mar. 14, 2013.

(51) Int. Cl. A61M 25/00; A61B 17/34

(52) U.S. Cl. CPC A61M 25/0021; A61B 17/3415
USPC 604/506; 604/239

(57) ABSTRACT

A regeneration device and method for sealing annular tears between the nucleus pulposus and the annulus fibrosus of an intervertebral disc. The regeneration device includes a trocar having a sharp tip at a distal end, an inner cavity, and a handle portion attached to a proximal end of the trocar. The device further includes a plunger coupled to a catheter that extends through the inner cavity. The regeneration method includes inserting the trocar into an intervertebral disc via Kambin's triangle, and once the trocar reaches the nucleus pulposus, the catheter is extended alongside the annulus fibrosus wall to surround the nucleus pulposus. A syringe is mounted to the plunger and injects a biochemical, biological and/or synthetic material through multiple ports in the catheter to form a collagen barrier that prevents the nucleus pulposus from protruding through the annulus fibrosus.

### CROSS REFERENCE TO RELATED APPLICATIONS

This application claims the benefit of the priority date of U.S. Provisional Patent Application No. 61/783,935, titled Percutaneous Intervertebral Annular Regeneration, filed Mar. 14, 2013.

### BACKGROUND OF THE INVENTION

1. Field of the Invention

This invention relates generally to a device and method for providing annular fibrosus regeneration of an intervertebral disc and, more particularly, to a device and method for providing annular fibrosus regeneration of an intervertebral disc using a biochemical, biological and/or synthetic material provided between the nucleus pulposus and the annulus fibrosus to seal annular tears.

2. Discussion of the Related Art

An intervertebral disc is a layer of cartilaginous material that stabilizes the spine and distributes forces between vertebrae. The intervertebral disc consists of an outer annulus fibrosus and an inner nucleus pulposus. The annulus fibrosus is made of strong lamellar layers of collagen fibers woven together at different angles, which provide withholding structures to the disc to keep the inner nucleus pulposis from herniating and allow for even force distribution among the surrounding vertebrae. The annulus fibrosus contains and protects the fluid, gel-like nucleus pulposus inside the disc. On the other hand, the nucleus pulposus consists of chondrocytes, collagen fibrils, and proteoglycan aggrecans which provide the capacity to bind water and support the adjacent vertebrae. The nucleus pulposus acts as a shock absorber, absorbing the impact of the body's daily activities while allowing movement of the adjacent vertebrae.

An intervertebral disc may be displaced or damaged due to trauma, disease, general wear and tear, or even age. Over time, the annulus fibrosus loses flexibility and elasticity; and the nucleus pulposus loses hydration, thus causing the interverterbral disc to lose height and leading to instability, tear or rupture. When a disc tears or ruptures, the disruption of the annulus fibrosis may allow the nucleus pulposus to bulge out into the vertebral canal, a condition commonly referred to as a herniated disc (also known as ruptured disc or slipped disc). The bulged out nucleus pulposus may press onto the spinal nerve and release inflammatory chemical mediators, which may result in nerve damage, pain, numbness, muscle weakness, and paralysis. If the herniated disc is in the lumbar region, the patient has a high possibility of experiencing sciatica that involves lower back and leg pain. Sciatica is the most common symptom of a lower back herniated disc, due to irritation of one of the nerve roots of the sciatic nerve while allowing movement of the adjacent vertebrae. Additionally, discogenic pain, i.e., pain originating from the intervertebral disc, can result in debilitating back pain even in a situation where there is no significant disc bulge or herniation. Patient's with discogenic back pain often experience debilitating pain and cannot work or perform activities of daily living.

A method that can be used to identify the source of intervertebral discogenic pain is a discography. In this procedure, dye is injected into the disc and the patient is asked whether he or she is experiencing a similar sort of back pain or not. This is followed by a computer tomography (CT) scan that shows if the injected dye is leaking out into the spinal canal. The leakage of dye is through tears in the annulus fibrosis. In many cases the patient's origin of pain is from the intervertebral disc level with concordant pain and annular tears, reflected by the egress of dye into the spinal canal as seen on the CT scan. These levels are usually treated by spinal fusion with removal of the intervertebral disc. In contrast, normal appearing discs do not generate much pain with injection of the dye, and show an intact annulus fibrosis, i.e., the dye stays within the center of the disc. It is believed that annular tears allow inflammatory factors to exit through the annulus and irritate the adjacent neural elements, and thus result in the back pain symptoms.

Herniated discs can occur in any part of the spine, but the majority of herniated disc occur in the lumbar region (lower back), while the second most common site is the cervical region (neck), and then the thoracic region (upper back) only accounts for a small percentage.

A herniated disc is often diagnosed through imaging, such as a magnetic resonance imaging (MRI) or a CT scan. In most cases, a herniated disc does not require surgery because it will heal on its own over time with rest. Approximately about half of the people with a herniated disc will improve within one month, while others will see improvement within six month. Despite the high improvement rates, about one in ten patients still requires surgery.

Initial non-surgical treatment serves to reduce pain and inflammation, and usually consists of non-steroidal anti-inflammatory pain medication; however, the long-term use of such medication may result in cardiovascular and gastrointestinal toxicity. An alternative treatment is to inject cortisone, a steroid hormone, into the spine to ease the pain. Nevertheless, these types of injection may still result in serious complications with long-term use. Other non-surgical treatments include physical therapy, chiropractic manipulation, anti-depressant, and other exercise programs.

If the condition continues to deteriorate, the last resort is surgery. The most common and effective surgery for herniated disc is a discectomy. A discectomy is the surgical removal of the herniated disc and more specifically, the nucleus pulposus that presses the nerve root or the spinal cord. Aside from the common surgical risks involving infection, anesthesia, and damages to the spine or nerves, the removal of the herniated disc may allow the disc space to collapse. A collapsed disc space will lead to instability of the spine, abnormal joint mechanics, nerve damage, and severe pain.

In recent years, intradiscal electrothermal annuloplasty (IDET), a minimally invasive form of herniated disc treatment, has been introduced. IDET includes the process of inserting a percutaneous threading of a flexible catheter into the herniated disc under fluoroscopic guidance. The catheter is composed of thermal resistive coil. Once the percutaneous catheter has reached the disc, the thermal resistive coil is heated to about 90° C. The heat shrinks and thickens the disc wall's collagen fibers for sealing any ruptures in the disc wall, and the heated coil may also used for burning nerve endings, which reduces pain by making the area less sensitive to pain. Studies have shown that IDET can relieve pain and improve mobility with an approximately 55% success rate, but the healing process takes time. Furthermore, the efficacy of IDET has not been assessed in randomized controlled clinical trials. This treatment technique has largely been abandoned due to poor clinical outcomes and non-reimbursement by insurance companies.

There is a need for treating a herniated disc less invasively and with a higher success rate while restoring the integrity of the annulus fibrosis and maintaining the nucleus pulposus.

### SUMMARY OF THE INVENTION

In accordance with the teachings of the present invention, a regeneration device and method for sealing annular tears between the nucleus pulposus and the annulus fibrosus of an intervertebral disc is disclosed. The regeneration device includes a trocar having a sharp tip at a distal end, an inner cavity, and a handle portion attached to a proximal end of the trocar. The device further includes a plunger coupled to a catheter that extends through the inner cavity. The regeneration method includes inserting the trocar into an intervertebral disc via Kambin's triangle, and once the trocar reaches the nucleus pulposus, the catheter is extended alongside the annulus fibrosus wall to surround the nucleus pulposus. A syringe is mounted to the plunger and injects a biochemical, biological and/or synthetic material through multiple ports in the catheter to form a collagen or collagen like barrier that prevents the nucleus pulposus from protruding through the annulus fibrosus.

Additional features of the present invention will become apparent from the following description and appended claims, taken in conjunction with the accompanying drawings.

### BRIEF DESCRIPTION OF THE DRAWINGS

FIGS. 1 and 2 are perspective views of a regeneration device;

FIG. 3 is a perspective view of the regeneration device percutaneously inserted into a disc;

FIG. 4 is a perspective view of the regeneration device with a catheter extending along an annulus fibrosus;

FIG. 5 is a perspective view of a syringe connected to the regeneration device to inject biochemical, biological and/or synthetic material through the catheter;

FIG. 6 is a close-up view of the catheter releasing a biochemical, biological and/or synthetic material along the annulus fibrosus; and

FIG. 7 is a close-up view of a biochemical, biological and/or synthetic barrier sealing annular tears along the annulus fibrosus.

### DETAILED DESCRIPTION OF THE EMBODIMENTS

The following discussion of the embodiments of the invention directed to a device and method for providing annular regeneration is merely exemplary in nature, and is in no way intended to limit the invention or its application or uses.

FIGS. 1 and 2 are perspective views of a disc regeneration device 10 including an outer trocar 16 that is used to puncture through an intervertebral disc, and an inner flexible catheter 22 having a plurality of strategically positioned ports 34 to administer a disc regeneration material, such as a biochemical, biological and/or synthetic material or stem cells. The trocar 16 includes a sharp tip 18 at a distal end and a handle portion 14 at a proximal end. The trocar 16 further includes an inner cavity 20 that allows the catheter 22 to extend through the trocar 16. The catheter 22 is also connected to and in fluid communication with a plunger 12 having a handle 42 and a rod 44 extending therefrom, where the rod 44 is attached to an end of the catheter 22 within the cavity 20. When the plunger 12 is advanced and the rod 44 is pushed into the cavity 20, the catheter 22 extends from the tip 18, and when the plunger 12 is retracted and the rod 44 is pulled from the cavity 20, the catheter 22 retracts into the cavity 20 through the tip 18.

FIG. 3 is a perspective view showing the tip 18 of the regeneration device 10 being percutaneously inserted into an intervertebral disc 24 adjacent to a vertebra 30 via Kambin's triangle. The insertion of the tip 18 may require the guidance of fluoroscopy, ultrasound, MRI, CT, image guidance or other imaging techniques. A radiopaque or echogenic coating on the trocar 16 and the catheter 22 enhances visual detection to ascertain device position within the intervertebral disc 24. When the trocar 16 reaches the nucleus pulposus 26 of the disc 24, the plunger 12 is pushed to extend the catheter 22 alongside an inside surface of the annulus fibrosus 28 of the disc 24.

FIG. 4 is a perspective view of the regeneration device 10 with the catheter 22 extending along the annulus fibrosus 28 within the disc 24. The catheter 22 is structurally flexible so that it is capable of completely surrounding the nucleus pulposus 26.

FIG. 5 is a perspective view of a syringe 32 connected to the regeneration device 10 to inject the biochemical, biological and/or synthetic material through the catheter 22, where the material may be a collagen material including chondrocytes. When the catheter 22 is fully extended, the syringe 32 containing the biochemical, biological and/or synthetic material is coupled to the plunger 12 through an end opening (not shown). The biological, biological and/or synthetic material is injected by the syringe 32 through a channel in the rod 44 into the catheter 22 where it is expelled as a biochemical, biological and/or synthetic material 36 into the nucleus pulposus 26 through the ports 34.

FIG. 6 is a close-up view of the catheter 22 releasing the biochemical, biological and/or synthetic material 36 along the annulus fibrosus 28 to form a seal or barrier 40 inside of the annulus fibrosus 28. When the syringe 32 pushes the biochemical, biological and/or synthetic material 36 into the catheter 22, the ports 34 allow the biochemical, biological and/or synthetic material 36 to form the barrier 40 that seals the nucleus pulposus 26 from protruding through the annulus fibrosus 28. FIG. 7 shows the barrier 40 formed inside of the annulus fibrosus 28 completely around the nucleus pulposus 26 when the catheter 22 is removed.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

What is claimed is:

1. A regeneration device for annular regeneration, said device comprising:
a trocar having one end including a sharp tip, an opposite end and an inner cavity therebetween;
a handle portion attached to the opposite end of the trocar;
a plunger mounted to the handle portion; and
a catheter having a plurality of ports coupled to the plunger and extended through the inner cavity of the trocar.

2. The regeneration device according to claim 1 wherein the catheter is in fluid communication with the plunger.

3. The regeneration device according to claim 1 wherein the catheter is controlled by the plunger.

4. The regeneration device according to claim 1 further comprising a syringe coupled to the plunger for injecting a biochemical, biological and/or synthetic material through the ports when the catheter is extended.

5. The regeneration device according to claim 4 wherein the material is a collagen material including chondrocytes.

6. The regeneration device according to claim 4 wherein the material includes stem cells.

7. The regeneration device according to claim 1 wherein the trocar includes a radiopaque or echogenic coating.

8. A regeneration device for percutaneous intervertebral annular disc regeneration, said device comprising:
a trocar including a distal end, a proximal end and a channel extending therebetween, said distal end having a sharp tip;
a handle mounted to the proximal end of the trocar;
a plunger including a rod extending through the handle and into the channel;
a flexible catheter positioned within the channel and being coupled to the rod, said catheter including a plurality of ports, wherein the rod operates to push the catheter through the channel so that a portion of the catheter including the ports extends out of the distal end of the trocar; and
a syringe coupled to the plunger and being in fluid communication with the catheter through the rod, said syringe providing a biochemical, biological and/or synthetic material to the catheter that flows out of the ports when the proximal end of the trocar is inserted into the disc.

9. The regeneration device according to claim 8 wherein the trocar includes a radiopaque or echogenic coating.

10. The regeneration device according to claim 8 wherein the material is a collagen material including chondrocytes.

11. The regeneration device according to claim 8 wherein the material includes stem cells.

12. A method for percutaneous intervertebral annular disc regeneration, said method comprising:
providing a trocar with a sharp tip at a distal end and an inner cavity;
providing a handle portion attached to a proximal end of the trocar;
providing a plunger mounted to the handle portion;
providing a catheter with multiple ports connected to the plunger and extended through the inner cavity;
inserting the tip of the trocar into an intervertebral disc;
extending the catheter within the intervertebral disc by pushing the plunger;
administering a biochemical, biological and/or synthetic material through ports in the catheter alongside an annulus fibrosus of the disc after the catheter is extended; and
removing the catheter from the intervertebral disc.

13. The method according to claim 12 wherein the catheter is in fluid communication with the plunger.

14. The method according to claim 12 wherein the plunger controls the catheter.

15. The method according to claim 12 further comprising mounting a syringe to the plunger for injecting the biochemical, biological and/or synthetic material after the catheter is fully extended.

16. The method according to claim 12 wherein inserting the tip of the trocar into an intervertebral disc includes inserting the tip via Kambin's triangle.

17. The method according to claim 12 wherein the catheter is flexible.

18. The method according to claim 12 wherein the material includes chondrocytes.

19. The method according to claim 12 wherein the material includes stem cells.
